(19) United States
(12) Patent Application Publication
(10) Pub. No.: US 2019/0172148 A1
(43) Pub. Date: Jun. 6, 2019

(54) Private Health Insurance Exchange System and Method

(71) Applicants: Carlos Samuel Garcia, Guaynabo, PR (US); Eduardo Zetina, Guaynabo, PR (US); Luis A. Medina, Guaynabo, PR (US); Carlos Garcia-Flores, Guaynabo, PR (US)

(72) Inventors: Carlos Samuel Garcia, Guaynabo, PR (US); Eduardo Zetina, Guaynabo, PR (US); Luis A. Medina, Guaynabo, PR (US); Carlos Garcia-Flores, Guaynabo, PR (US)

(21) Appl. No.: 16/239,962

(22) Filed: Jan. 4, 2019

Related U.S. Application Data
(63) Continuation-in-part of application No. 14/297,119, filed on Jun. 5, 2014.

(51) Int. Cl. G06Q 40/08; G16H 10/60

(52) U.S. Cl. CPC G06Q 40/08; G16H 10/60

(57) ABSTRACT

Systems and methods for the distribution, equalization and mitigation of risks in an insurance exchange, and specifically to that of a private health insurance exchange system and method that rewards/punishes those attempting to poach or game it. The system and method also includes a way to create an insurance exchange so that those providers participating may get all or a fraction of any losses that exceed pre-established limits reimbursed to them as part of a payment by those providers participating in the exchange who received gains exceeding their expectations.

### CROSS-REFERENCE TO RELATED APPLICATIONS

This application claims priority as a Continuation-In-Part of co-pending U.S. patent application Ser. No. 14/297,119 titled “Private Health Insurance Exchange System and Method”, filed on Jun. 5, 2014 the disclosure of which is herein incorporated by reference in its entirety.

### FIELD OF THE INVENTION

The present invention relates to a system and method for the distribution, equalization and mitigation of risks in an insurance exchange, and specifically to that of a private health insurance exchange system and method that rewards/punishes those attempting to poach or game it.

### DESCRIPTION OF THE RELATED ART

The United States spends more on healthcare than any other nation in the world as a portion of gross domestic product. Although the Affordable Care Act makes significant progress in terms of access and fairness in the insurance marketplace, there is concern that the fundamental causes of rising costs have not been addressed. Instead of a single payer system, the present and foreseeable setup will still force employers to receive bids from two or more suppliers in order to be assured of value through competition.

In effect, to reduce risks, costs and provide more options, employers often opt to have two or more insurance providers bid to provide insurance services to a member group. In many cases, the providers to a single group (such as a large employer), proceed to form an insurance pool.

When establishing such a pool to cover a group of members, insurance service providers need to estimate the expected usage of services by the member's (be they individual's, families or any other subgroup) usage of the services. If the provider underestimates, then the carrier or company will expend significantly more resources than those billed, losing money. If they overestimate, then their rates will be too high, and consumers (say the employees of said client company) will go with another member of the insuring pool.

The above in effect means that those providing the expensive coverage, are bound to have only employees with many expenses, reducing the pool of healthy members necessary to distribute the risks. But the natural fragmentation of covered insurance groups creates the possibility of insurance carriers (also called insurance companies) ‘gaming the system’ in order to subscribe only healthy adults.

What is needed is a way to create an insurance exchange so that those providers participating may get all or a fraction of any losses that exceed pre-established limits reimbursed to them as part of a payment by those providers participating in the exchange who received gains exceeding their expectations.

### SUMMARY OF THE INVENTION

This section is for the purpose of summarizing some aspects of the present invention and to briefly introduce some preferred embodiments. Simplifications or omissions may be made to avoid obscuring the purpose of the section. Such simplifications or omissions are not intended to limit the scope of the present invention.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein; this reference does not constitute an admission that any of these documents form part of the common general knowledge in the art.

In one aspect the invention is about a health exchange and risk-sharing system comprising at least one computer processor, a non-transitory computer-readable medium embodying a program executable by said at least one computer processor, the program when executed causing said at least one computer processor to do the following: confirming, by said at least one computer processor, an establishment of an inter-carrier agreement to share and blend risk associated with an employer-sponsored group health plan among two or more participating insurance carriers, collecting, by said at least one computer processor, data and evaluating the performance of one or more of each said participating insurance carrier over a period of time as defined by the inter-carrier agreement to share and blend risk, calculating, by said at least one computer processor, medical loss ratios (MLR's) for each said participating insurance carrier, determining an overall blended or combined MLR between participating insurance carriers, determining a risk share-adjusted MLR for each said participating insurance carrier, and determining an experience adjustment factor for each participating insurance carrier and calculating, by said at least one computer processor, through the use of the calculate MLR's for each said participating insurance carrier, the overall blended or combined MLR between the participating insurance carriers, thus being able to calculate and determine the financial obligations or rewards for each said participating insurance carrier based on the pre-agreed experience adjustment factor that has been agreed to by each participating insurance carrier. In another aspect, said pre-agreed adjusted experience factor is used in the assessment of financial rewards or obligations. In yet another aspect, each of said n carrier MLR (MLRₙ) is calculated by taking each said carrier's loss (CLₙ) and dividing it by each said carrier's premium (Prₙ), generating a combined MLR (MLR_{comb}) based on the following formula: MLR_{comb}=(CL₁+CL₂+ . . . +CLₙ)/(Pr₁+Pr₂+ . . . +Prₙ), said pre-agreed adjusted experience factor (W) is agreed upon by all carriers before generation of said MLR_{comb}, an effective MLR for each said carriers (MLR_{EFFn}) is calculated using the following formula: MLR_{EFFn}=(W)*(MLR_{comb})+(1−W)*(MLRₙ) and a financial adjustment (FAₙ) for each carrier n is calculated using the following formula: FAₙ=Prₙ*(MLRₙ−MLR_{EFFn}).

In one aspect, the invention is about a computer-implemented health exchange and risk-sharing method comprising confirming, by a computer server, an establishment of an inter-carrier agreement to share and blend risk associated with an employer-sponsored group health plan among two or more participating insurance carriers, collecting, by the computer server, data and evaluating the performance of one or more of each said participating insurance carrier over a period of time as defined by the inter-carrier agreement to share and blend risk, calculating by the computer server, medical loss ratios (MLR's) for each said participating insurance carrier, determining the overall blended or combined MLR between participating insurance carriers, determining the risk share-adjusted MLR for each said participating insurance carrier, and determining the experience adjustment factor for each participating insurance carrier and calculating, by said at least one computer processor, through the use of each said MLR's for each said participating insurance carrier, the overall blended or combined MLR between the participating insurance carriers, thus being able to calculate and determine the financial obligations or rewards for each participating insurance carrier, based on their performance of their individual MLR against said blended or combined MLR. In another aspect a pre-agreed adjusted experience factor is used in the assessment of financial rewards or obligations. In yet another aspect, each of said n carrier's MLR (MLRₙ) is calculated by taking each said carrier's loss (CLₙ) and dividing it by each said carrier's premium (Prₙ), generating a combined MLR (MLR_{comb}) based on the following formula: MLR_{comb}=(CL₁+CL₂+ . . . +CLₙ)/(Pr₁+Pr₂+ . . . +Prₙ); an adjusted experience factor (W) is agreed upon by all carriers based on said generated MLR_{comb}, an effective MLR for each said carrier (MLR_{EFFn}) is calculated using the following formula: MLR_{EFFn}=(W)*(MLR_{comb})+(1−W)*(MLRₙ); and a financial adjustment (FAₙ) for each carrier n is calculated using the following formula FAₙ=Prₙ*(MLRₙ−MLR_{EFFn}).

In one aspect, the invention is about a non-transitory computer-readable medium embodying a program executable by at least one computing device, the program when executed causing said at least one computing device to do the following, confirming, by said at least one computing device, an establishment of an inter-carrier agreement to share and blend risk associated with an employer-sponsored group health plan among two or more participating insurance carriers, collecting, by said at least one computing device, data and evaluating the performance of one or more of each said participating insurance carrier over a period of time as defined by the inter-carrier agreement to share and blend risk, calculating, by said at least one computing device, medical loss ratios (MLR's) for each said participating insurance carrier, determining the overall blended or combined MLR between participating insurance carriers, determining the risk share-adjusted MLR for each said participating insurance carrier, and determining the experience adjustment factor for each participating insurance carrier and calculating, by said at least one computer processor, through the use of each said MLR's for each said participating insurance carrier, the overall blended or combined MLR between the participating insurance carriers, thus being able to calculate and determine the financial obligations or rewards for each participating insurance carrier, based on their performance of their individual MLR against said blended or combined MLR. In another aspect, a pre-agreed adjusted experience factor is used in the assessment of financial rewards or obligations. In yet another aspect each of said n carriers MLR (MLRₙ) is calculated by taking each said carrier's loss (CLₙ) and dividing it by each said carrier's premium (Prₙ), generating a combined MLR (MLR_{comb}) based on the following formula: MLR_{comb}=(CL₁+CL₂+ . . . +CLₙ)/(Pr₁+Pr₂+ . . . +Prₙ), an adjusted experience factor (W) is agreed upon by all carriers based on said generated MLR_{comb}, an effective MLR for each said carrier (MLR_{EFFn}) is calculated using the following formula, MLR_{EFFn}=(W)*(MLR_{comb})+(1−W)*(MLRₙ) and a financial adjustment (FAₙ) for each carrier n is calculated using the following formula FAₙ=Prₙ*(MLRₙ−MLR_{EFFn}).

Other features and advantages of the present invention will become apparent upon examining the following detailed description of an embodiment thereof, taken in conjunction with the attached drawings.

### BRIEF DESCRIPTION OF THE DRAWINGS

FIGS. 1-2 show illustrations of the advantage of an Exchange and Risk Sharing, according to exemplary embodiments of the invention.

FIG. 3 shows an exemplary Bronze level premium pricing table, according to an illustrative embodiment of the invention.

FIG. 4 shows various computing components of the system, according to an illustrative embodiment of the invention.

FIG. 5 shows a typical competitive scenario between three carriers (A, B, C), according to an illustrative exemplary embodiment of the invention.

FIGS. 6-10 show examples of five different scenarios for five carriers (A, B, C, D, E) funds distributions using the Riskshare® system and method, according to an exemplary embodiments of the invention.

The above-described and other features will be appreciated and understood by those skilled in the art from the following detailed description, drawings, and appended claims.

### DETAILED DESCRIPTION OF THE PREFERRED EMBODIMENT

To provide an overall understanding of the invention, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

Simplifications or omissions may be made to avoid obscuring the purpose of the section. Such simplifications or omissions are not intended to limit the scope of the present invention. All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

As used in the specification and claims, the singular forms “a”, “an” and “the” include plural references unless the context clearly dictates otherwise. For example, the term “a transaction” may include a plurality of transaction unless the context clearly dictates otherwise. As used in the specification and claims, singular names or types referenced include variations within the family of said name unless the context clearly dictates otherwise.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the modules or any assembly of them may be used.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Alternate Solution to Mitigate Adverse Selection in Multi-Carrier Insurance Programs. Statement of the Technical Problem: One of the biggest challenges facing any insurance program is ensuring that the members that ultimately participate in the program are an adequate cross-section representation of the population that was the subject of the risk analysis performed to determine premium rates. In a single-carrier scenario, this is accomplished by requiring minimum participation levels (e.g. 75% of eligible employees). However, in a multi-carrier scenario minimum participation levels alone are inadequate since they do not address the polarization of risk that occurs as higher risk individuals gravitate toward higher priced more comprehensive plans and lower risk individuals often select less comprehensive, more economical plans.

This effect, known as Adverse Selection, poses a particularly difficult technical problem for pricing plans. If the more comprehensive plan tries to build in the greater concentration of high-risk individuals into its prices it runs the risk of exacerbating the problem by further concentrating high risk beyond expectation, as high prices tend to turn off a greater proportion of otherwise healthy individuals than would have participated were the plan's prices lower. On the other hand, if the plan tries to artificially lower its rates to appeal to a wider customer base, it runs the risk of not generating enough premium revenue to cover the medical expenses of the portion of the population that selected that plan. Conversely, the less comprehensive plan typically generates much more revenue than would be required to cover the claims expense of its segment of the population—a population characterized by a lower than normal concentration of high-risk individuals.

When looking at the current industry solution, we see that in order to try and remedy the situation described above that occurs in a multi-carrier health insurance program, the industry has developed something called Risk Adjustment. There are many risk adjustment systems in place across the industry, but they all share some common features. All seek to assign ‘risk scores’ to each and every individual participating in the program, usually based on submitted diagnostic codes, in an effort to try and measure the average morbidity of the segments of the population that ultimately selected each participating carrier. These systems then assign a dollar value to the differences in expected morbidity so that premium can be clawed back from carriers whose average risk scores fall below the population average, while at the same time paying that premium back out to those carriers whose average risk scores fell above the population average.

Generally speaking risk adjustment systems are very time-consuming and typically require very large populations to improve their effectiveness. But ultimately these systems have two fundamental flaws. First, risk-adjusted premium adjustments are not based on how much was actually spent on patient care by each plan, but rather on the diagnostic codes submitted with claims data, usually from periods prior to the one being adjusted. Second, an artificial incentive is created to up-code medical services rendered as competing plans try to portray their segment of the population as the one with the most serious medical conditions. Plans redirect a tremendous amount of resources to this practice of ‘harvesting’ diagnostic codes, rather than managing care directly.

The above leads to seeing the proposed RiskShare® as an Alternate Solution. The RiskShare® methodology replaces the risk adjustment system with a much more straightforward and practical solution to the technical problem of adverse selection. RiskShare® recognizes that the true goal of premium adjustment in a multi-carrier program is to better cover the claims risk associated with each carrier's segment of the population.

RiskShare® solves the problem of adverse selection by calculating weighted average MLRs (Medical Loss Ratios) based on each carrier's actual MLR and on the blended aggregate MLR (Combined MLR) that results from the premium and claims experience of all participating plans combined, all without harvesting a single diagnostic code. This simplified methodology simultaneously addresses both fundamental flaws of risk adjustment systems. First, by using MLRs as the basis for adjustment rather than diagnostic codes, RiskShare® focuses adjustment based strictly on how much was actually spent by each carrier's segment of the patient population, regardless of how the medical services rendered were coded for diagnoses. Second, because MLRs are used and not diagnostic codes, there is virtually no incentive for plans to chase high-value codes in an effort to game the system. Because of its simplicity and its singular focus on adjusting dollar amounts based on what was actually spent, the RiskShare® methodology is a far superior solution to the technical problem of adverse selection in multi-carrier health insurance programs. Further, the RiskShare® methodology makes multi-carrier programs feasible in much smaller populations than would be required for application of risk adjustment systems.

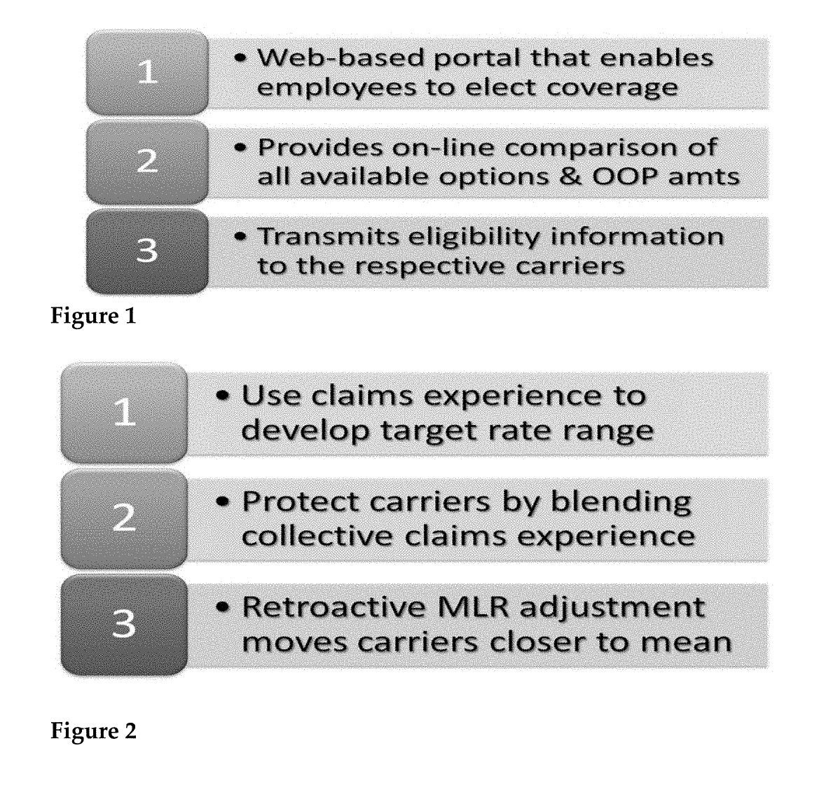

Referring to FIGS. 1-2, we see exemplary embodiments for illustrations of the advantage of an Exchange and Risk Sharing flowchart for the embodiment of both an exchange and a RiskShare© Methodology system and method. Through such an arrangement, a health exchange operator or agent could pool one or more insurance providers so that in one embodiment, all group rates to be quoted by Health Insurance Carrier/Company(s) to a client follow a Uniform Rate Structure (URS) with fixed ratios for Individuals, Couples and Family tiers (or any other acceptable subgroup within the insured population for which a price structure is defined).

In one embodiment, imagine that fixed ratios are determined for Bronze, Silver and Gold plan designs or levels (or any other suitable stratification), so that fixed ratios for Medical, Vision, Drug and Dental rate components may be calculated, thus allowing for fixed ratios for rate levels between sub-groups within the plan's group clients as a whole. Such an URS allows ALL rates to be defined by one single Core Rate (CR), which is then to be determined individually by each Health Insurance Company (HIC) participating in the exemplary exchange.

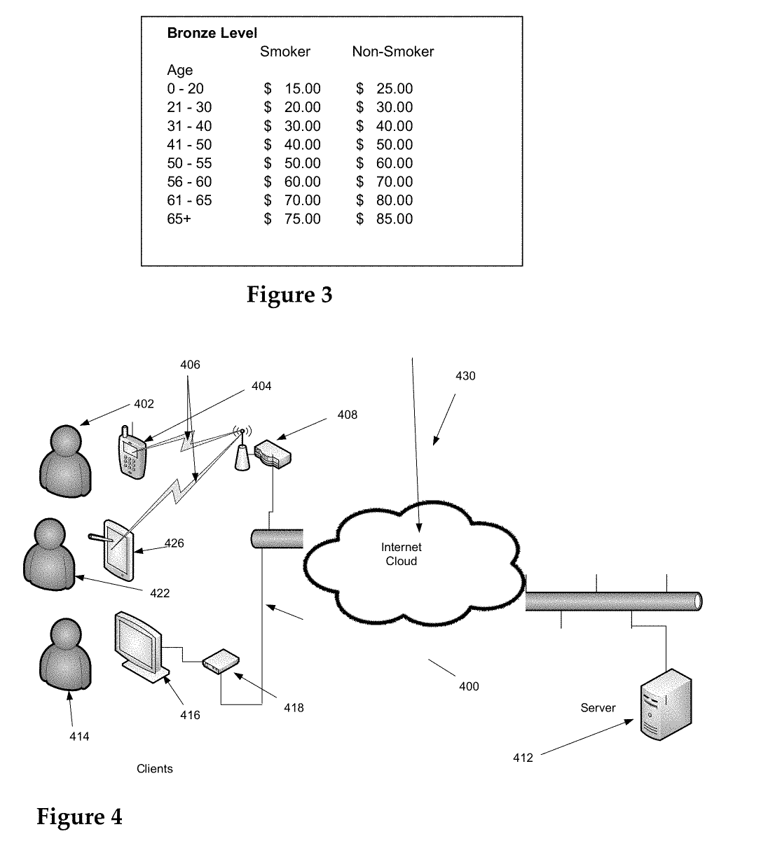

The above designs or levels, i.e. Gold/Silver/Bronze, are typically based on a number of risk factors and the expected expenses for an individual within them. In practice, these are usually regulated by the person's date of birth and whether they are smokers or not, but a large number of other factors may be taken into account in their calculation. As seen in FIG. 3, an exemplary table describes the monthly premium for the Bronze level plan, based on age of the client and whether they are a smoker or non-smoker. Similar tables would be available for Silver and Gold levels, either for combined Health/Dental/Vision/Drug or separately.

In one embodiment, a period of time evaluation of a particular length is selected (typically a year, but any suitable length of time may be used, from one year to multi years). The system then proceeds to take into account the least twelve months of claims experience, using appropriate estimates for incurred but unpaid claims.

That is, the exchange operator (in this case an entity we will name Qinetix) performs a Common Group Rating Exercise to produce THREE (3) sets of rates for each underwritten group: (a) a Target Rate Set, (b) a Lower Bound Rate Set and (c) an Upper Bound Rate Set, each with a corresponding Core Rate that produces the given set. Clearly in alternate embodiments, any number of said sets of rates may be generated, as well as any number of underwritten groups or segmentations performed. Participating Health Insurance Companies are then encouraged to submit a Core Rate that produces a rate set similar to the Target Rate Set, although of course HICs are free to submit what they feel is required to run a profitable carrier.

The freedom of HICs to submit a Core Rate above or below the produced or generated Target Rate Set level provides a possibility that carriers may decide to ‘buy’ some business, that is, underbid a particular Core Rate and ‘make it up in volume’. To discourage such underbidding and ‘gaming’ of the system (whereupon a participating supplier or insurer underbids everyone in order to “make it up in volume”). In effect, the exchange requires that rates quoted BELOW the Lower Bound Rate Set will alter the RiskShare© formula, as described below.

As part of the enrollment process via the Private Health Insurance Exchange, each HIC will ultimately enroll only a subset of the entire group client's employees and dependents. Since the Common Group Rating Exercise is based on the group client's claims experience as a whole, it is likely that underwriting Medical Loss Records (MLR) results will vary significantly from HIC to HIC. The purpose of the RiskShare© Methodology is to significantly mitigate such deviations from the overall mean.

The agent or exchange operator, in this example Qinetix, will then become responsible for analyzing and determining the Consolidated MLR, comprised of one of at least each HIC's Company MLR (including premium adjustments, if any, as described), each HIC's Settled Company MLR and finally each HIC's Final Settlement Amount.

In addition to the final settlement calculation, Qinetix will also provide its best estimate of projected settlement amounts in order for Health Insurance Companies to properly accrue payables or receivables at key financial period closes. To prevent the “gaming” of the system, the exchange operator (in this case Qinetix) would generate a recommended minimum premium, based on the various parameters related to statistical and other insurance metrics. Any carrier/company bidding a price below said number, would have its basis MLR affected inversely by the amount underbid. In effect, a penalty is paid when ‘buying business to make it up in volume”.

In one embodiment, the HICs or providers would provide the clients’ employees (such as an employer or group) various insurance options, to be purchased by said employees through said Private Health Insurance Exchange. In such a system, the coverage offered through the different insurance products adheres to those stated on each provider's proposal. In this fashion, each HIC establishes the pricing schedule as stated on its proposal, while attaching an APPENDIX for each health insurance product, according to the rating structures and guidelines provided in the customer's or exchange provider RFP.

The employees then individually select, purchase and process their health insurance application through the Private Health Insurance Exchange (PHIX), which in one embodiment is an internet portal through which the employees log on, in order to select and process their health insurance applications. They may do this through their Smartphone, tablet, desktop computer or any other similar device connected to the Internet.

Referring to FIG. 4, we illustrate various exemplary embodiments of user interactions with the PHIX system 400. In one embodiment, a user 402 uses the resident application in his Smartphone 404 or another user 422 uses a Tablet 426 to link through the wireless or mobile internet 408 (be it LTE, Wi-Fi or other such similar wireless link) 406 to the system backbone (which may be an internet-type link, Virtual Private network or similar) to a bridge 408 through the internet cloud 410 to one or more servers 412 hosting the PHIX exchange computer component. Similarly, another user 414 may use a traditional browser on a desktop computer 416 through a cable modem 418 and fiber/wired high speed connection 420 to the cloud 410, hence to the servers 412.

The devices shown may operate Apple OS, Android, Linux, Windows and other such operating systems, and be comprised of desktops, tablets, Smartphones (e.g. iPhones, iPads, Nexus, Samsung and other devices), with the application being an App, Browser or otherwise driven. The Private Health Insurance Exchange provides each employee all available health insurance options along with a monthly payment calculator for each option based on the agreed upon employer contributions.

The operator of the exchange, say Qinetix, uses the herein described methodology and systems in order to ensure better access to health insurance for employees while mitigating exposure to risk for Health Insurance Companies. As a condition for offering their products through the Private Health Insurance Exchange, each HIC agrees to participate in the RiskShare© Methodology, with the exchange operator being responsible for managing the RiskShare© Methodology in order secure full compliance on the part of each HIC. The parties understand that the RiskShare© Methodology will include a claims experience adjustment process that may result in financial disbursement between the parties.

In one embodiment, each HIC presents a guarantee bond in order to guarantee its participation and fulfillment of its responsibilities in the RiskShare© Methodology. This guarantee bond represents in one embodiment ten percent (10%) of the total amount of annualized premiums to be underwritten by each HIC, although it may include higher/lower numbers based on credit and previous performance.

In one embodiment, the PHIX system operator provides a number of services, including; technical support during the health insurance purchasing process to both employees and producers; process and forward the health insurance applications filed electronically by employees as part of the health insurance purchasing process; Manage the RiskShare© Methodology; and Secure full compliance of all Health Insurance Companies with the terms and conditions of the Private Health Insurance Exchange and the RiskShare© Methodology.

Based on the aggregate premium and claims data from all Health Insurance Companies combined, Qinetix calculates a Consolidated MLR (Medical Loss Ratio). This is a well understood term in the medical insurance business, which defines what percentage of the paid claims was paid out.

As an example, an MLR of 80% means the insurer paid out 80% of the paid dues in claims (so they made money), whereas one of 120% means they lost money by paying out more than they took in. The claims data will consider claims incurred during the group contract period (twelve months), plus four additional months of run-out claims as paid by each participating HIC. In alternate embodiments, any chosen period of time may be used.

Premium data will consider Earned Premium (enrollment-based premium), regardless of whether said premium has been collected or not by each HIC. In the case of HIC(s) that opted to quote a Core Rate that produced a rate set BELOW the Lower Bound Rate Set, an adjustment will be made to said HIC's premium data to simulate the premium that would have been earned had the Lower Bound Rate Set been billed.

The Consolidated MLR is then be calculated by dividing the aggregate claims data by the aggregate premium data, adjusted as described above. Using the same considerations for claims data and premium data described above, a Company MLR will be calculated for each participating HIC. The RiskShare© Methodology consists of an artificial adjustment (the Final Settlement Amount) to each HIC's claims expense with the purpose of moving each HIC's Company MLR closer to the Consolidated MLR.

Generally, if a given Company MLR (after any premium adjustment) is ABOVE the Consolidated MLR, then the given HIC will receive funds to offset its claims expense, thereby improving its adverse MLR results. Conversely, if a given Company MLR (after any premium adjustment) is BELOW the Consolidated MLR, then the given HIC will remit funds to other participating Health Insurance Companies, resulting in an artificial increase to its recognized claims expense and its ultimate MLR results.

In one embodiment, the magnitude of the artificial adjustment in claims expense (the Final Settlement Amount), whether positive or negative, will be equal to an amount required to alter one HIC's MLR (after any premium adjustment) to a Settled Company MLR equal to the weighted average MLR based on 75% weight applied to the Consolidated MLR and 25% weight applied to the HIC's MLR (after any premium adjustment). Note that in alternate embodiments, any appropriate percentage distribution may be used.

As an example, consider a company participating in the exchange which allows two HICs (Companies A and B) to offer plans to their 1,000 employees. Half of the employees (500) select company A, while the remaining 500 select company B. After one year, Carrier A has premiums of $1.0M, claims of $700K, for an MLR of 70%. Company B had the same premiums $1.0M, but claims of $900K, resulting in an MLR of 90%. In effect, the employees subscribed to B had more claims, i.e. they got sick more often.

Because they were part of the PHIX operated by Qinetix, both companies agreed a priory to share the risk, as well as to a pre-agreed adjusted experience factor. The overall combined experience is a premium of $2.0M ($1 MM for each company), claims of $1.6M ($900K+$700K), which generate a combined MLR of 80%. As such, the pre-agreed adjusted experience factor comes into play. While this pre-agreed ratio may be any ratio, in one embodiment both providers agreed to use an adjusted experience factor of 75% (as the weight towards blended). As seen in Table 1 and Table 2 below, the abeyance by the two or more participants in the schema to a pre-approved experience ratio allows for the sharing of the risks within a fair assessment

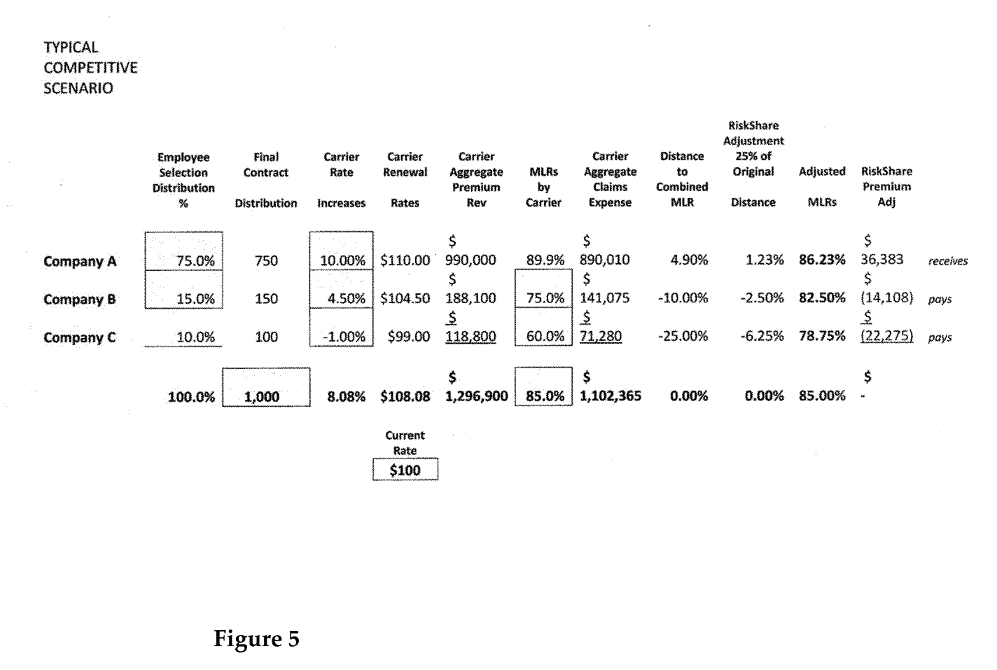

Because of the above, in one embodiment, the combined MLR is reduced/increased by said number. Thus, the effective MLR of company A after the adjusted experience would be 77.5% MLR, and that of company B that of 82.5% MLR. As such, company A would transfer $75 K to company B. FIG. 5 illustrates an example for a Qinetix Corporate Exchange in a typical competitive scenario, wherein we see the numbers and Riskshare® methodology result for three separate companies (A, B, C).

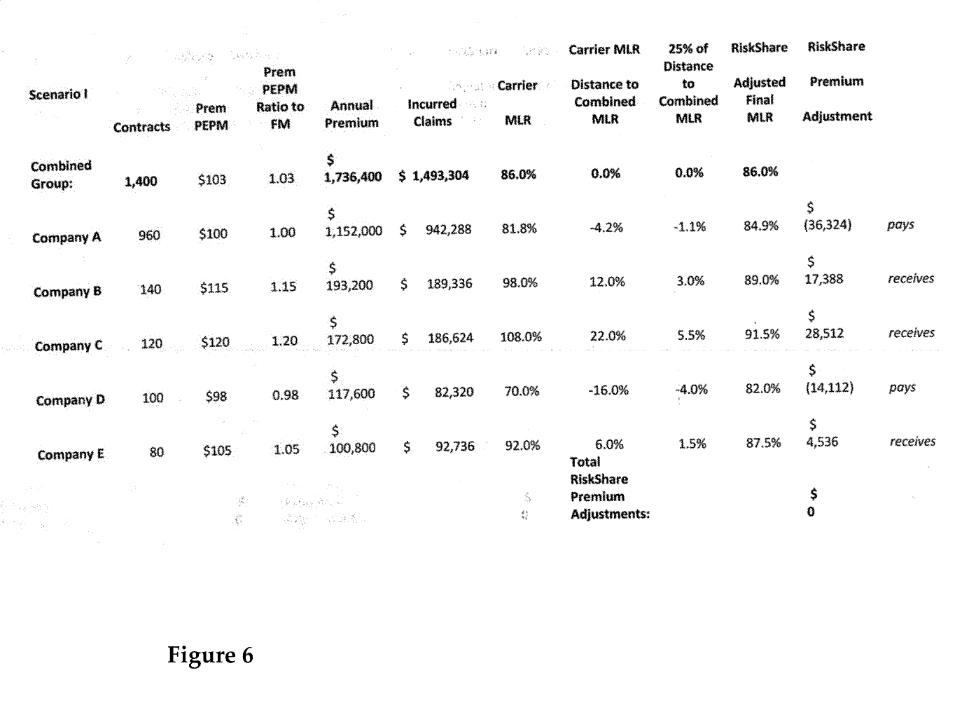

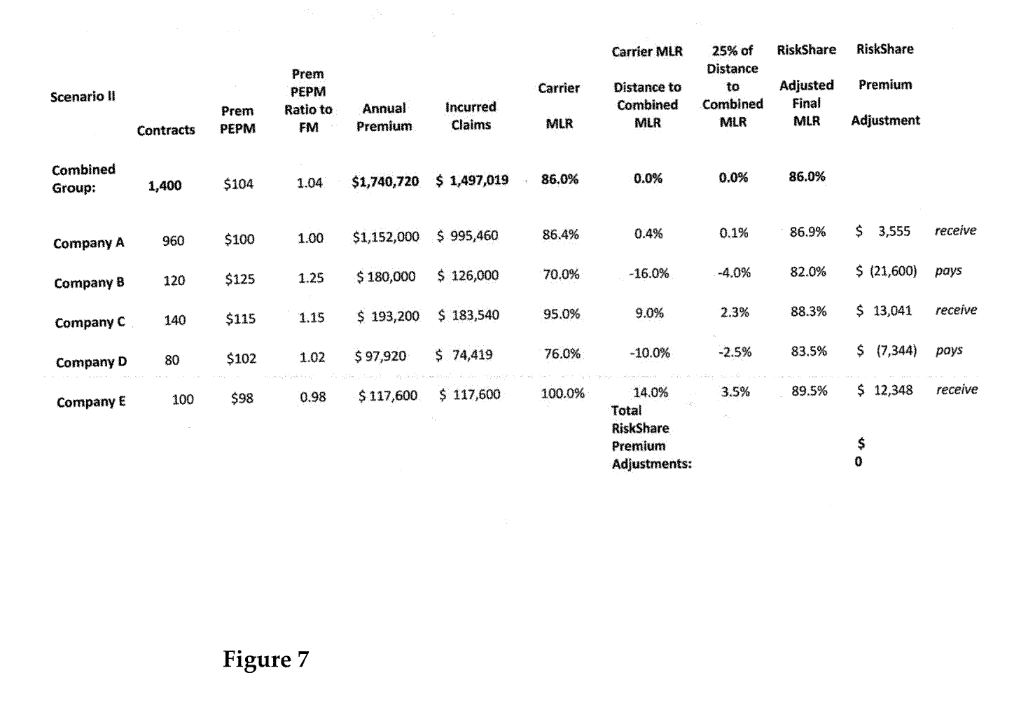

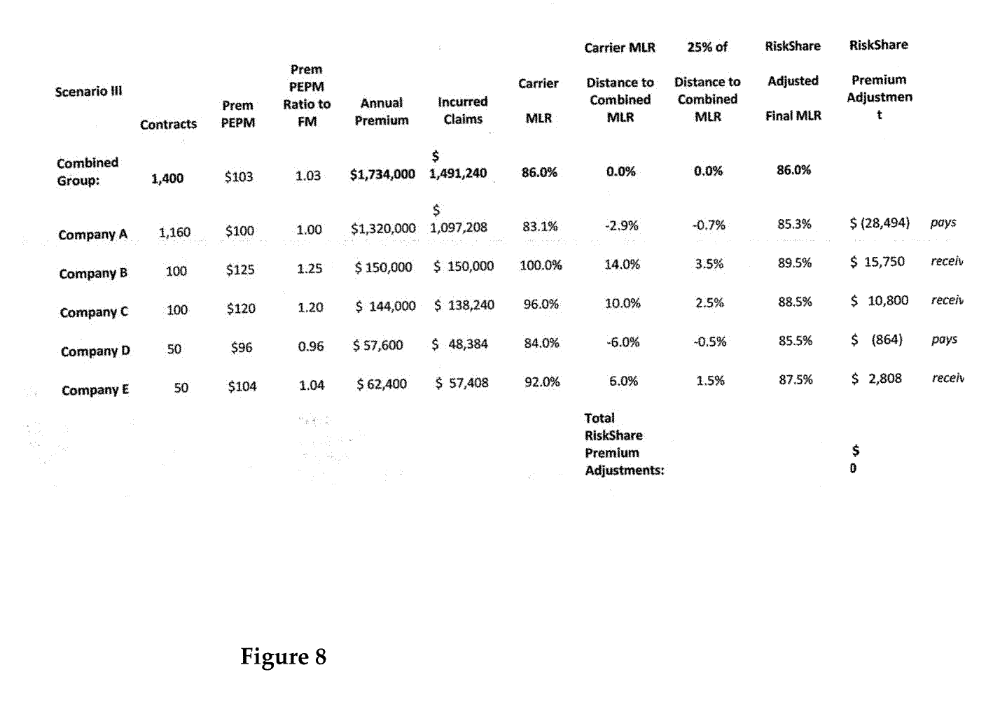

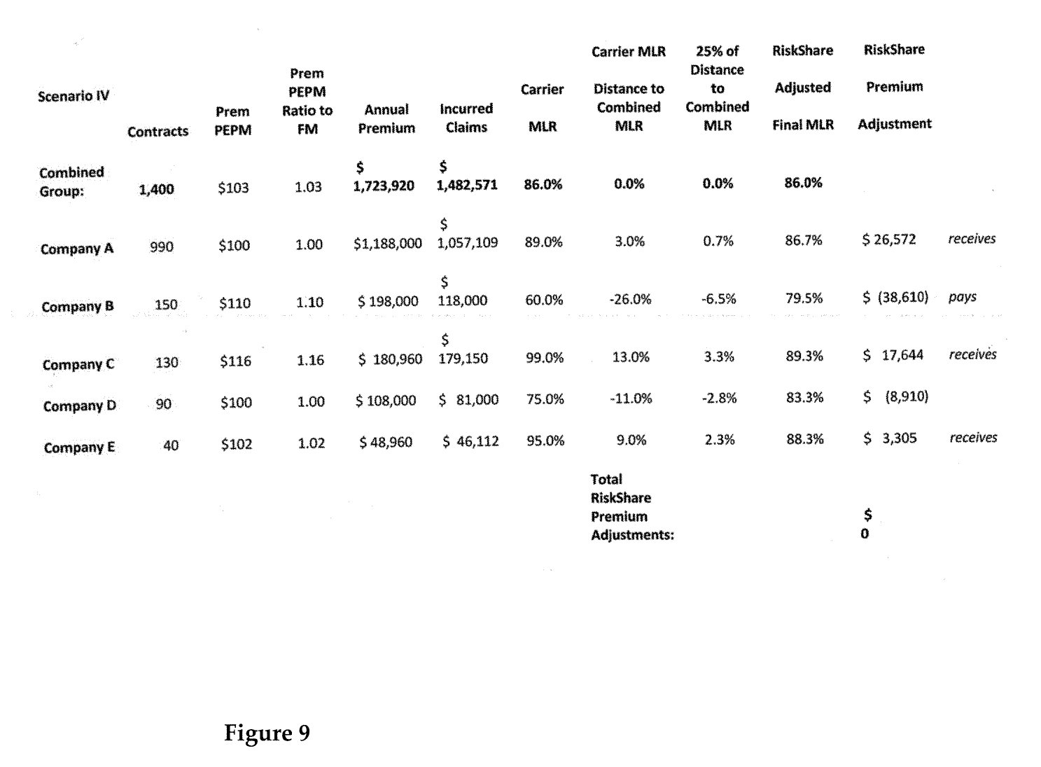

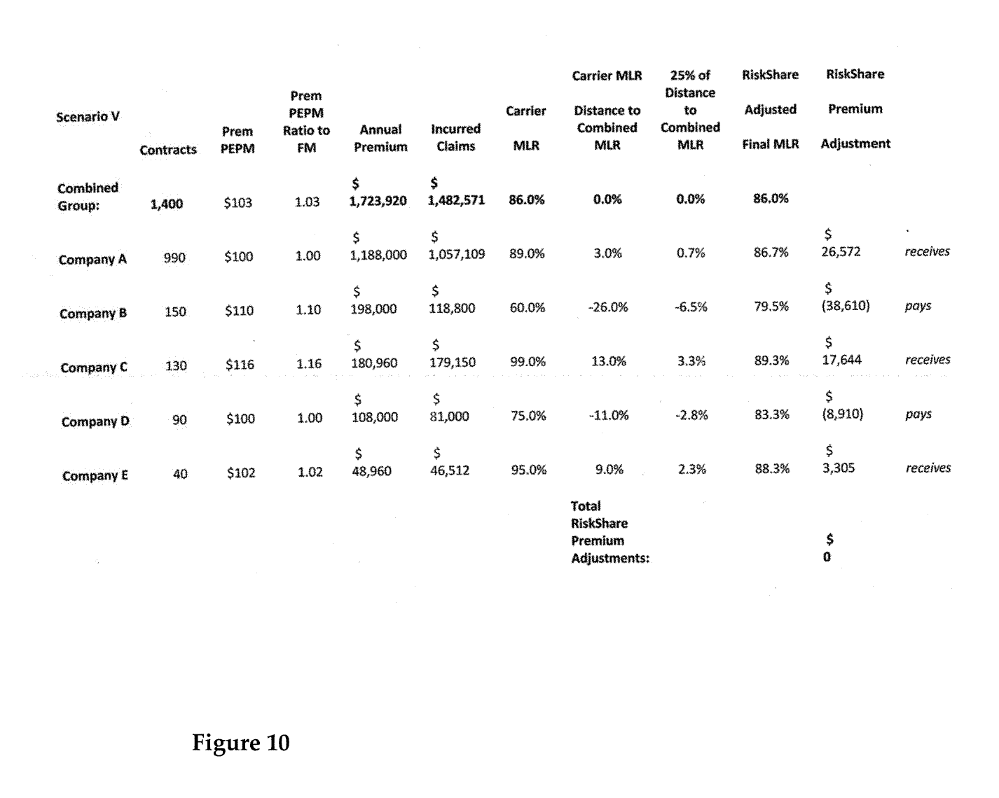

Similarly, FIGS. 6-10 illustrate large group exchanges under five separate scenarios based on the premise that the overall MLR (as if the entire group were with ONE single carrier) for the 2014-2015 contract period, for the sake of these scenarios, is assumed to be 86%. The actual Combined MLR will of course be above or below this assumption. However, for purposes of modeling different scenarios, we use 86.0% MLR as the assumed base Combined MLR.

The Final Settlement Amounts will then be communicated within 60 days (or any other appropriate amount of time) of the close of the four-month claims run-out period. Health Insurance Companies will have an additional period of time (say 30, 45, 60 or any suitable number of days) from the date an invoice for Final Settlement, if any, is received. Each HIC is required to procure a guarantee bond, as described in the Risk-Sharing Agreement, to ensure compliance with Final Settlement liabilities.

Example 1 below, further illustrates the process. In general, all group rates to be quoted by Health Insurance Companies follow a Uniform Rate Structure with fixed ratios for Individual, Couple and Family tiers; fixed ratios for PLAN NAME 1, PLAN NAME 2 and PLAN NAME 3 plan designs; fixed ratios for Medical, Drug and Dental rate components; and fixed ratios for rate levels between sub-groups of the group client as a whole. This Uniform Rate Structure allows ALL rates to be defined by one single Core Rate to be determined individually by each Health Insurance Company.

Based on at least twelve months of claims experience with appropriate estimates for incurred but unpaid claims, Qinetix will perform a Common Group Rating Exercise to produce THREE (3) sets of rates for each underwritten group: (a) a Target Rate Set, (b) a Lower Bound Rate Set and (c) an Upper Bound Rate Set, each with a corresponding Core Rate that produces the given set.

While Health Insurance Companies are encouraged to submit a Core Rate that produces a rate set similar to the Target Rate Set, Health Insurance Companies are certainly at liberty to submit a Core Rate above or below that Target Rate Set level. However, it is important to note that quoting a Core Rate that produces rates BELOW the Lower Bound Rate Set alters the Riskshare® methodology formula, as described below.

As a result of the enrollment process via the Private Health Insurance Exchange, each Health Insurance Company will ultimately enroll only a subset of the entire group client's employees and dependents. Since the Common Group Rating Exercise is based on the group client's claims experience as a whole, it is likely that underwriting (MLR) results will vary significantly from Health Insurance Company to Health Insurance Company. The purpose of the Riskshare® methodology is to significantly mitigate such deviations from the overall mean.

Based on the aggregate premium and claims data from all Health Insurance Companies combined, Qinetix will calculate a Consolidated MLR (Medical Loss Ratio). The claims data will consider claims incurred during the group contract period (twelve months), plus six additional months of run-out claims as paid by each participating Health Insurance Company. Premium data will consider Earned Premium (enrollment-based premium), regardless of whether premium has been collected or not by each Health Insurance Company. In the case of Health Insurance Companies that opted to quote a Core Rate that produced a rate set BELOW the Lower Bound Rate Set, an adjustment will be made to said Health Insurance Company's premium data so as to simulate the premium that would have been earned had the Lower Bound Rate Set been billed. The Consolidated MLR will then be calculated by dividing the aggregate claims data by the aggregate premium data, adjusted as described above.

Using the same considerations for claims data and premium data described above, a Company MLR will be calculated for each participating Health Insurance Company.

The Riskshare® methodology consists of an artificial adjustment (the Final Settlement Amount) to each Health Insurance Company's claims expense with the purpose of moving each Health Insurance Company's Company MLR closer to the Consolidated MLR. Generally, if a given Company MLR (after any premium adjustment) is ABOVE the Consolidated MLR, then the given Health Insurance Company will receive funds to offset its claims expense, thereby improving its adverse MLR results. Conversely, if a given Company MLR (after any premium adjustment) is BELOW the Consolidated MLR, then the given Health Insurance Company will remit funds to other participating Health Insurance Companies, resulting in an artificial increase to its recognized claims expense and its ultimate MLR results.

The magnitude of the artificial adjustment in claims expense (the Final Settlement Amount), whether positive or negative, will be equal to an amount required to alter the Company MLR (after any premium adjustment) to a Settled Company MLR equal to the weighted average MLR based on 75% weight applied to the Consolidated MLR and 25% weight applied to the Company MLR (after any premium adjustment).

Qinetix will be responsible for analyzing and determining the Consolidated MLR, each Health Insurance Company's Company MLR (including premium adjustments, if any, as described above), each Health Insurance Company's Settled Company MLR and finally each Health Insurance Company's Final Settlement Amount. In addition to the final settlement calculation, Qinetix will also provide its best estimate of projected settlement amounts in order for Health Insurance Companies to properly accrue payables or receivables at key financial period closes.

In one embodiment, each Health Insurance Company agrees to accept and abide by the Final Settlement Amount, as analyzed and calculated by Qinetix, and to remit payments, if necessary. A Final Settlement will be communicated within 60 days of the close of the six-month claims run-out period. Health Insurance Companies will have an additional 30 days from the date an invoice is received for the payment of the Final Settlement Amounts, if applicable.

TABLE 1

| | | | | [symbol] from |
|---|---|---|---|---|
| | Premium | Claims | MLR | Combined |
| (A) | $500,000 | $400,000 | 80% | +1.25% |
| (B) | $1,000,000 | $1,050,000 | 105% | +26.25% |
| (C) | $700,000 | $350,000 | 50% | −28.75% |
| (D) | $800,000 | $600,000 | 75% | −3.75% |
| (E) | $200,000 | $120,000 | 60% | −18.75% |
| Comb. | $3,200,000 | $2,520,000 | 78.75% | 0.00% |

TABLE 1

| | | Exp. Adj. Factor | Exp. Adj. Factor | Adjusted |
|---|---|---|---|---|
| | 25% of [symbol] | (−75% of [symbol] ) | Applied to Prem. | Claims |
| (A) | +0.3125% | −0.9375% | ($4,687.50) | $395,312.50 |
| (B) | +6.5625% | −19.6875% | ($196,875.00) | $853,125.00 |
| (C) | −7.1875% | +21.5625% | $150,937.50 | $500,937.50 |
| (D) | −0.9375% | +2.8125% | $22,500.00 | $622,500.00 |
| (E) | −4.6875% | +14.0625% | $28,125.00 | $148,125.00 |
| | | | 0.00 | $2,520,000.00 |

### CONCLUSION

In concluding the detailed description, it should be noted that it would be obvious to those skilled in the art that many variations and modifications can be made to the preferred embodiment without substantially departing from the principles of the present invention. Also, such variations and modifications are intended to be included herein within the scope of the present invention as set forth in the appended claims. Further, in the claims hereafter, the structures, materials, acts and equivalents of all means or step-plus function elements are intended to include any structure, materials or acts for performing their cited functions.

It should be emphasized that the above-described embodiments of the present invention, particularly any “preferred embodiments” are merely possible examples of the implementations, merely set forth for a clear understanding of the principles of the invention. Any variations and modifications may be made to the above-described embodiments of the invention without departing substantially from the spirit of the principles of the invention. All such modifications and variations are intended to be included herein within the scope of the disclosure and present invention and protected by the following claims.

The present invention has been described in sufficient detail with a certain degree of particularity. The utilities thereof are appreciated by those skilled in the art. It is understood to those skilled in the art that the present disclosure of embodiments has been made by way of examples only and that numerous changes in the arrangement and combination of parts may be resorted without departing from the spirit and scope of the invention as claimed. Accordingly, the scope of the present invention is defined by the appended claims rather than the forgoing description of embodiments.

1. A health exchange and risk-sharing system comprising:
at least one computer processor;
a non-transitory computer-readable medium embodying a program executable by said at least one computer processor, the program when executed causing said at least one computer processor to do the following:
confirming, by said at least one computer processor, an establishment of an inter-carrier agreement to share and blend risk associated with an employer-sponsored group health plan among two or more participating insurance carriers;
collecting, by said at least one computer processor, data and evaluating the performance of one or more of each said participating insurance carrier over a period of time as defined by the inter-carrier agreement to share and blend risk;
calculating, by said at least one computer processor, consolidated medical loss ratios (MLR's) for each said participating insurance carrier, determining an overall blended or combined MLR between participating insurance carriers, determining a risk share-adjusted MLR for each said participating insurance carrier based on the pre-agreed experience adjustment factor that has been agreed to by each participating insurance carrier; and
calculating, by said at least one computer processor, through the use of the calculated MLR's for each said participating insurance carrier, the overall blended or combined MLR between the participating insurance carriers, thus being able to calculate and determine the financial obligations or rewards for each participating insurance carrier, based on the performance of their individual MLR against said blended or combined MLR.

2. The health exchange system of claim 1 wherein:
said pre-agreed adjusted experience factor is used in the assessment of financial rewards or obligations.

3. The system of claim 1 wherein:
each of said n carrier MLR (MLRₙ) is calculated by taking each said carrier's loss (CLₙ) and dividing it by each said carrier's premium (Prₙ), generating a calculated MLR (MLR_{comb}) based on the following formula:
MLR_{comb}=(CL₁+CL₂+ . . . +CLₙ)/(Pr₁+Pr₂+ . . . +Prₙ);
said pre-agreed adjusted experience factor (W) is agreed upon by all carriers before generation of said MLR_{comb};
an effective MLR for each said carrier (MLR_{EFFn}) is calculated using the following formula:
MLR_{EFFn}=(W)*(MLR_{comb})+(1−W)*(MLRₙ); and
a financial adjustment (FAₙ) for each carrier n is calculated using the following formula:
FAₙ=Prₙ*(MLRₙ−MLR_{EFFn}).

4. A computer-implemented health exchange and risk-sharing method comprising:
confirming, by a computer server, an establishment of an inter-carrier agreement to share and blend risk associated with an employer-sponsored group health plan among two or more participating insurance carriers;
collecting, by the computer server, data and evaluating the performance of one or more of each said participating insurance carrier over a period of time as defined by the inter-carrier agreement to share and blend risk;
calculating by the computer server, consolidated medical loss ratios (MLR's) for each said participating insurance carrier, determining the overall blended or combined MLR between participating insurance carriers, determining the risk share-adjusted MLR for each said participating insurance carrier based on the pre-agreed experience adjustment factor that has been agreed to by each participating insurance carrier; and
calculating, by said at least one computer processor, through the use of each said calculated MLR's for each said participating insurance carrier, the overall blended or combined MLR between the participating insurance carriers, thus being able to calculate and determine the financial obligations or rewards for each participating insurance carrier, based on their performance of their individual MLR against said blended or combined MLR.

5. The method of claim 4 wherein;
said pre-agreed adjusted experience factor is used in the assessment of financial rewards or obligations.

6. The method of claim 4 wherein;
each of said n carriers MLR (MLRₙ) is calculated by taking each said carrier's loss (CLₙ) and dividing it by each said carrier's premium (Prₙ), generating a consolidated MLR (MLR_{comb}) based on the following formula:
MLR_{comb}=(CL₁U+CL₂+ . . . +CLₙ)/(Pr₁+Pr₂+ . . . +Prₙ);
said pre-agreed adjusted experience factor (W) is agreed upon by all carriers before generation of said MLR_{comb};
an effective MLR for each said carrier (MLR_{EFFn}) is calculated using the following formula:
MLR_{EFFn}=(W)*(MLR_{comb})+(1−W)*(MLRₙ); and
a financial adjustment (FAₙ) for each carrier n is calculated using the following formula:
FAₙ=Prₙ*(MLRₙ−MLR_{EFFn}).

7. A non-transitory computer-readable medium embodying a program executable by at least one computing device, the program when executed causing said at least one computing device to do the following:
confirming, by said at least one computing device, an establishment of an inter-carrier agreement to share and blend risk associated with an employer-sponsored group health plan among two or more participating insurance carriers;
collecting, by said at least one computing device, data and evaluating the performance of one or more of each said participating insurance carrier over a period of time as defined by the inter-carrier agreement to share and blend risk;
calculating, by said at least one computing device, consolidated medical loss ratios (MLR's) for each said participating insurance carrier, determining the overall blended or combined MLR between participating insurance carriers, determining the risk share-adjusted MLR for each said participating insurance carrier based on the pre-agreed experience adjustment factor that has been agreed to by each participating insurance carrier; and
calculating, by said at least one computer processor, through the use of each said calculated MLR's for each said participating insurance carrier, the overall blended or combined MLR between the participating insurance carriers, thus being able to calculate and determine the financial obligations or rewards for each participating insurance carrier, based on their performance of their individual MLR against said blended or combined MLR.

8. The method of claim 7 wherein;
said pre-agreed adjusted experience factor is used in the assessment of financial rewards or obligations.

9. The non-transitory computer-readable medium embodying a program executable in at least one computing device of claim 7 wherein;
each of said n carriers MLR (MLRₙ) is calculated by taking each said carrier's loss (CLₙ) and dividing it by each said carrier's premium (Prₙ), generating a combined MLR (MLR_{comb}) based on the following formula:
MLR_{comb}=(CL₁+CL₂+ . . . +CLₙ)/(Pr₁+Pr₂+ . . . +Prₙ);
said pre-agreed adjusted experience factor (W) is agreed upon by all carriers before generation of said MLR_{comb};
an effective MLR for each said carrier (MLR_{EFFn}) is calculated using the following formula:
MLR_{EFFn}=(W)*(MLR_{comb})+(1−W)*(MLRₙ); and
a financial adjustment (FAₙ) for each carrier n is calculated using the following formula:
FAₙ=Prₙ*(MLRₙ−MLR_{EFFn}).
